(19) United States
(12) Patent Application Publication
(10) Pub. No.: US 2024/0238555 A1
(43) Pub. Date: Jul. 18, 2024

(54) GUIDE EXTENSION CATHETER

(71) Applicant: Teleflex Life Sciences LLC, Wilmington, DE (US)

(72) Inventors: Christopher E. Buller, Toronto (CA); Joshua Brenizer, Oak Grove, MN (US); Dean Peterson, Minneapolis, MN (US); Allison Margulies, Minneapolis, MN (US)

(73) Assignee: Teleflex Life Sciences LLC, Wilmington, DE (US)

(21) Appl. No.: 18/412,217

(22) Filed: Jan. 12, 2024

Related U.S. Application Data
(60) Provisional application No. 63/479,612, filed on Jan. 12, 2023.

(51) Int. Cl. A61M 25/00; A61M 25/06

(52) U.S. Cl. CPC A61M 25/0023; A61M 25/0012; A61M 25/005; A61M 25/0662; A61M 2025/0024

(57) ABSTRACT

A guide extension catheter positionable within a guide catheter and configured to receive an interventional device for insertion into vasculature is disclosed. The guide extension catheter can comprise a push member, a first reinforcement member in contact with the push member, and a radially-collapsible, tubular membrane in contact with the push member and the first reinforcement member. The tubular membrane can be positioned distal to the first reinforcement member and radially collapsed or wrapped about the push member prior to receiving the interventional device. The push member can provide column strength sufficient to prevent longitudinal collapse of the tubular membrane during use. The guide extension catheter can provide a low friction, large diameter (for a given compatible guide catheter size) pathway proximal to, and optionally through, a target lesion in the vasculature, thereby reducing micro and macro vasculature injury attributable to delivery of interventional devices. Related systems and methods are also disclosed.

### CROSS-REFERENCE TO RELATED APPLICATIONS

This application claims the benefit of priority under 35 U.S.C. § 119(c) to U.S. Provisional Appln. Ser. No. 63/479,612, filed Jan. 12, 2023, which is incorporated herein by reference in its entirety.

### TECHNICAL FIELD

The subject matter of this disclosure relates to the field of medical devices. Implementations relate to guide extension catheters and components thereof.

### BACKGROUND

The present disclosure relates generally to devices, systems, and methods for interventional procedures and more particularly to a guide extension catheter for aiding in the delivery of interventional devices to a treatment site within a patient.

In general, interventional procedures require delivering interventional devices though guide catheters. It is often necessary to deliver the interventional device to a desired location beyond a distal end of the guide catheter, i.e., a target tissue area, for the device to administer an effective treatment. However, delivery of the interventional device beyond the guide catheter may require high delivery force and can cause micro and/or macro injuries to vasculature en route to the target tissue area.

### SUMMARY

The present inventors recognize that there exists a need for catheter delivery devices, systems, and methods which can be used to deliver an interventional device to a desired location and shield the vasculature from abrasion or injury.

According to some embodiments, a guide extension catheter positionable within a guide catheter and configured to receive an interventional device for insertion into vasculature can comprise a push member, a first reinforcement member in contact with the push member, and a radially-collapsible, tubular membrane in contact with the push member and the first reinforcement member. The tubular membrane can be positioned distal to the first reinforcement member and collapsed or wrapped about the push member prior to receiving the interventional device.

According to some embodiments, a guide extension catheter for use with a guide catheter can comprise a radially-collapsible, tubular membrane defining a lumen, the lumen including a central axis, and a push member in contact with the tubular membrane along its entire length and extending proximal of the tubular membrane for slidably positioning the tubular membrane within and partially beyond a distal end of the guide catheter. The tubular membrane can have no effective radial strength and can be configured to collapse radially inward toward the central axis. The tubular membrane can include a tensile strength sufficient to prevent tearing during insertion of an interventional cardiology device.

According to some embodiments, a method for accessing a coronary artery can comprise providing a guide catheter, advancing the guide catheter through a blood vessel to a position adjacent to an ostium of the coronary artery, and providing a guide extension catheter. The guide extension catheter can include a push member and a radially-collapsible, tubular membrane wrapped about the push member prior to receiving an interventional device. The method can further comprise advancing the guide extension catheter through the guide catheter to a position where at least a portion of the tubular membrane extends distally beyond a distal end of the guide catheter and into the coronary artery, and advancing an interventional cardiology device through the guide catheter and into a lumen defined by the tubular membrane, including urging the tubular membrane to expand from a collapsed, wrapped configured to an expanded configuration.

These and other examples and features of the present devices, systems, and methods will be set forth, at least in part, in the following Detailed Description. This Overview is intended to provide non-limiting examples of the present subject matter—it is not intended to provide an exclusive or exhaustive explanation. The Detailed Description below is included to provide further information about the present devices, systems, and methods.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 illustrates a plan view of a guide catheter advanced through an aorta to an ostium of a coronary vessel, according to some embodiments.

FIG. 2 illustrates a plan view of a guide extension catheter, as constructed in accordance with at least one embodiment, used in conjunction with a guide catheter for the delivery of an interventional device into an occluded vessel for treatment.

FIG. 3 illustrates an isometric view of a guide catheter and a guide extension catheter having a fixed push member, according to some embodiments.

FIG. 4 illustrates an isometric view of a guide catheter, a guide extension catheter, and a guidewire, according to some embodiments.

FIG. 5A illustrates an isometric view of a guide extension catheter having a first reinforcement member, according to some embodiments.

FIG. 5B illustrates an isometric view of a guide extension catheter having a second reinforcement member, according to some embodiments.

FIG. 6A illustrates a cross-sectional view of a tube member with a push member disposed on an outer surface of the tube member, according to some embodiments.

FIG. 6B illustrates a cross-sectional view of a tube member with a push member disposed on an inner surface of the tube member, according to some embodiments.

FIG. 6C illustrates a cross-sectional view of a tube member with a push member disposed within a plane of the tube member, according to some embodiments.

FIG. 6D illustrates a cross-sectional view of a tube member and a guidewire with a push member disposed within a plane of the tube member, according to some embodiments.

FIG. 7A illustrates a cross-sectional view of a radially-collapsible, tubular membrane in an expanded configuration, according to some embodiments.

FIG. 7B illustrates a cross-sectional view of a radially-collapsible, tubular membrane folding pattern, according to some embodiments.

FIG. 7C illustrates a cross-sectional view of a radially-collapsible, tubular membrane folding pattern, according to some embodiments.

FIG. 7D illustrates a cross-sectional view of a radially-collapsible, tubular membrane folding pattern, according to some embodiments.

FIG. 8A illustrates a plan view of an interventional device advancing through a guide catheter and a guide extension catheter, according to some embodiments.

FIG. 8B illustrates a plan view of an interventional device advancing through a guide catheter and a guide extension catheter, according to some embodiments.

FIG. 8C illustrates a plan view of an interventional device advanced through a guide catheter and a guide extension catheter, according to some embodiments.

FIG. 9A illustrates an isometric view of a guide catheter and a guide extension catheter having a distal reinforcement member, according to some embodiments.

FIG. 9B illustrates a magnified isometric view of a distal portion of the guide extension catheter shown in FIG. 9A, according to some embodiments.

FIG. 9C illustrates a side view of an interventional device being retracted proximally toward a distal end of a configuration of the guide extension catheter shown in FIG. 9A, according to some embodiments.

FIG. 9D illustrates a side view of an interventional device being retracted proximally toward a distal end of another configuration of the guide extension catheter shown in FIG. 9A, according to some embodiments.

FIG. 10A illustrates an isometric view of a guide catheter and a guide extension catheter having a tube member of variable cross-sectional diameter, according to some embodiments.

FIG. 10B illustrates an en face view of a first configuration of a portion of the tube member shown in FIG. 10A, according to some embodiments.

FIG. 10C illustrates an en face view of a second configuration of the portion of the tube member shown in FIG. 10A, according to some embodiments.

FIG. 11 illustrates an isometric view of a guide catheter and a guide extension catheter having inflatable reinforcement members, according to some embodiments.

FIG. 12A illustrates a diagrammatic view of a column force acting on a tube member, according to some embodiments.

FIG. 12B illustrates a diagrammatic view of a bend force acting on a tube member, according to some embodiments.

FIG. 12C illustrates a diagrammatic view of a radial compressive force acting on a tube member, according to some embodiments.

FIG. 12D illustrates a diagrammatic view of a tensile or expansive force acting on a tube member, according to some embodiments.

FIG. 13 illustrates a flowchart of a method for accessing a coronary artery and providing treatment to the artery, according to some embodiments.

### DETAILED DESCRIPTION

According to some embodiments, this disclosure relates to a guide extension catheter having a push member, a reinforcement portion, and a radially-collapsible, tubular membrane having a lumen. The tubular membrane may have minimal effective radial strength in compression, minimal effective column strength, and minimal effective bend stiffness. The tubular membrane may have sufficient tensile strength to avoid tearing during insertion of an interventional device and during its removal from a patient. The tubular membrane may be durably lubricious on an inner surface to facilitate advancement and withdrawal of interventional devices through its lumen and may be durably lubricious on an outer surface to enhance delivery of the guide extension catheter into a blood vessel.

According to some embodiments, the push member may serve as a backbone to the tubular membrane. The push member may optionally be in the form of a guidewire or a gradually tapering push rod to help steer and support delivery of the guide extension catheter to a target tissue area. The reinforcement portion of the guide extension catheter may be disposed at a proximal end of the tubular membrane and may provide structural support to keep the proximal end of the tubular membrane's lumen open and accessible. The guide extension catheter may further include a second reinforcement portion disposed at a distal end of the tubular membrane, which is configured to keep the distal end of the tubular membrane's lumen open.

Devices, systems, and methods herein relate generally to delivery of medical treatment devices through a guide extension catheter, and more specifically to devices, systems, and methods for enhanced and atraumatic delivery of interventional devices in patients undergoing percutaneous interventions in order to (i) deliver interventional devices that may not easily be delivered with a chosen in-situ guide catheter alone, and/or (ii) reduce micro and macro arterial injury attributable to delivery of inflexible or non-lubricious interventional devices and existing guide extension catheters. It should be noted that although the description below is primarily directed toward cardiovascular percutaneous interventions, the devices, systems, and methods described herein may be used in other medical specialties, e.g., peripheral vasculature treatments, urinary treatments, respiratory treatments, digestive treatments, diagnostic endoscope treatments, and/or any other medical treatments that can benefit from the use of guide extension catheters.

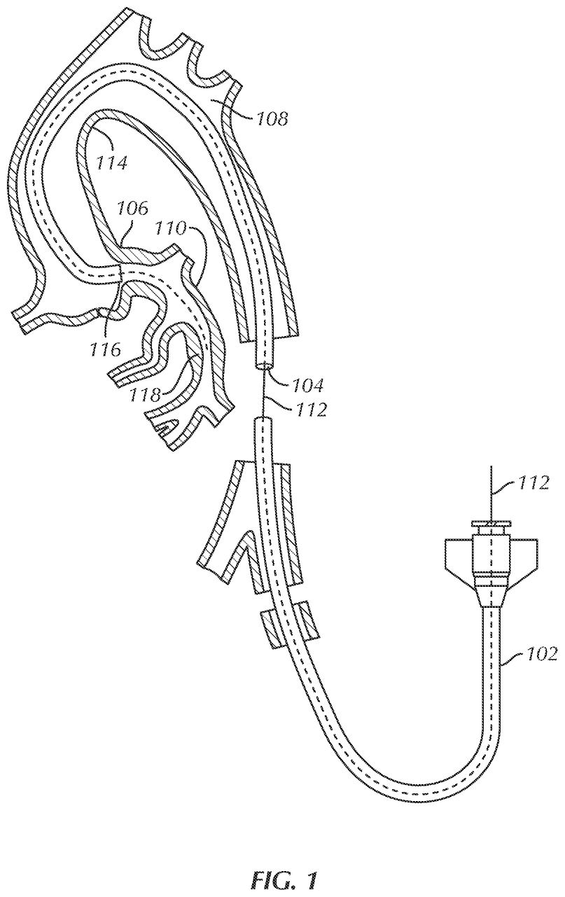

FIG. 1 illustrates an exemplary minimally invasive cardiac intervention, including a guidewire 112 and a guide catheter 102. The guidewire 112 can comprise an elongate, small-diameter member designed to navigate vessels to reach a diseased site or vessel segment of interest. Guidewires can come in various configurations, including stainless steel or nitinol core wires and/or solid core wires wrapped in a smaller wire coil, for example. The guide catheter 102 can comprise an elongate tube member defining a main lumen 104 along its length. The guide catheter 102 can be formed of polyurethane, for example, and can be shaped along its distal portion to facilitate advancement to and alignment with a coronary ostium 106 (or other region of interest within a patient's body). Various sized guide catheters 102, e.g., 6 F, 7 F, or 8 F guide catheter, where F is an abbreviation for the French catheter scale (a unit to measure catheter diameter (1 F=⅓ mm)), can be inserted at a femoral or radial artery and advanced through an aorta 108 to a position adjacent the ostium 106 of a coronary artery 110.

The guidewire 112 (or a shorter, thicker introducer guidewire) and guide catheter 102 can be advanced through the arch 114 of the aorta 108 to the ostium 106. The guidewire 112 may then be advanced beyond the ostium 106 and into the coronary artery 110. The diameter and rigidity of the guide catheter's distal end 116, however, may not permit the device to be safely advanced beyond the ostium 106 into the coronary artery 110.

Maintaining the position of the guide catheter's distal end 116 at the ostium 106 can facilitate the guidewire 112, or another interventional device, successfully reaching the diseased site (e.g., a stenotic lesion 118). With the guide catheter 102 in position, force can be applied to the guidewire's proximal end to push the guidewire 112 to and beyond the lesion 118, and a treating catheter (optionally including a balloon or stent) can be passed over the guidewire 112 to treat the site. The application of force to the guidewire 112 or the treating catheter can sometimes cause the guide catheter 102 to dislodge from the ostium 106 of the coronary artery 110, and, in such instances, the guidewire or treating catheter must be distally advanced independently of the guide catheter's ostial alignment and support to reach the lesion 118. This can occur in the case of a tough stenotic lesion 118 or tortuous anatomy, for example, where it is often difficult to pass the guidewire 112 or the treating catheter to and beyond the lesion. A heart's intrinsic beat can also cause the guide catheter's distal end 116 to lose its ostial positioning or otherwise be shifted so that it no longer is positioned to align and support the guidewire 112 or the treating catheter into the portion of the coronary artery 110 including the lesion 118.

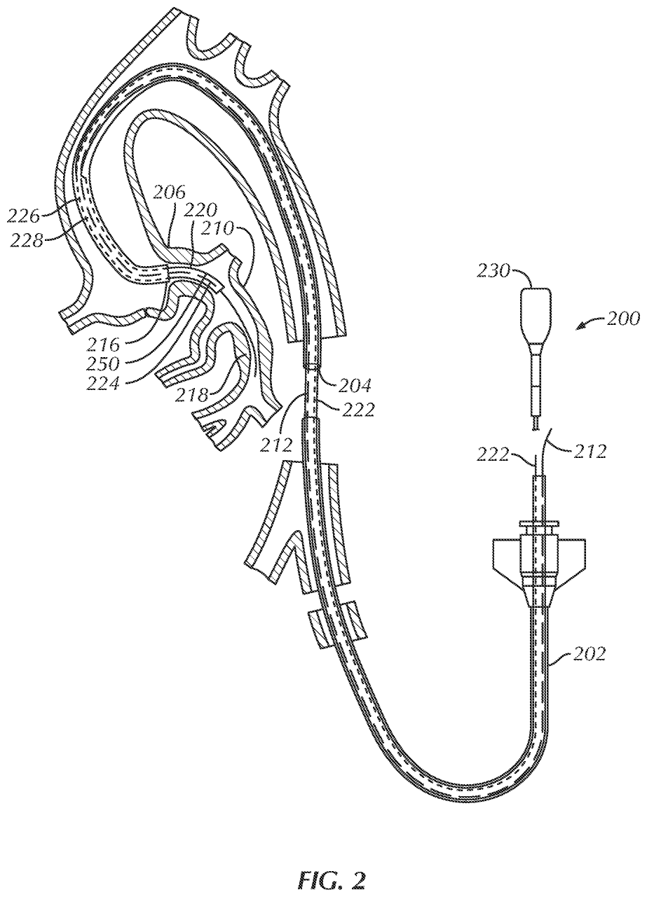

As first illustrated in FIG. 2, the present guide extension catheter 200 can improve access and provide protection to a coronary artery 210 leading up to, and optionally beyond, a stenotic lesion 218. The guide extension catheter 200 may include an elongate tube member 220 and a push member 222 having a collective length that is greater than a length of a guide catheter 202 (e.g., 130 cm-175 cm, or greater). An outer diameter of the tube member 220 can be sized to permit insertion of its distal end 224 through a guide catheter 202 and into the coronary artery 210 or its branches containing the lesion 218, thereby providing alignment, support, and a low friction pathway for an interventional device (e.g., a treating catheter) beyond the distal end 216 of the guide catheter 202 to, and optionally through, the lesion 218. The extension of the tube member 220 into a smaller-sized artery or branch can also serve to maintain the position of the guide catheter 202 at the artery's ostium 206 during an operation.

The push member 222 may be in the form of a guidewire or a gradually tapering push rod, for example, to help steer and support delivery of the guide extension catheter 200 to the lesion 218. The push member 222 may comprise a stainless steel, nitinol, or another substantially rigid material and can be configured to be sufficiently rigid in torque to avoid helical twisting of the guide extension catheter 200 during use. For example, the push member 222 may be flattened in cross section along one or more portions of its length to contribute to resistance to twisting and reduce a crossing profile of the guide extension catheter 200.

The tube member 220 may include a first reinforced portion (not shown) disposed at its proximal end 226 and a second reinforced portion disposed at its distal end 224. The tube member 220 may further include a soft, flexible, radially-collapsible tubular membrane 250 disposed distally to the first reinforced portion and proximally to the second reinforced portion.

The delivery of inflexible or non-lubricious interventional devices through a segment of the coronary artery 210 distal to the guide catheter 202 in the absence of the guide extension catheter 200 can produce (i) endothelial injury (micro injury) and may contribute to atheroembolism and type-4 periprocedural myocardial infarction, and/or (ii) more serious macro injuries including plaque disruption and coronary dissection leading to acute/threatened ischemic complication—any of which can contribute to atherosclerosis progression and eventual target-vessel failure. The soft, flexible, radially-collapsible tubular membrane 250 can reduce device-artery interactions by providing a thin-walled structure that lines the artery and provides a lubricious intra-coronary delivery pathway.

In some embodiments, the operating physician can advance the distal end portion 224 of the tube member 220 over a guidewire 212 and through and beyond the guide catheter's distal end 216 into the coronary artery 210 by applying a longitudinal force to the push member 222 directly or via a handle member 230, such as the handle member 230 described in commonly-owned U.S. Pat. Pub. No. 2019/0247619, which is hereby incorporated by reference in its entirety. The handle member 230 may include a flexible clip or clamp configured to attach to an external object when not being moved, as described in commonly-owned U.S. Pat. Pub. No. 2021/0008342, which is hereby incorporated by reference in its entirety. The proximal end portion 226 of the tube member 220 may remain within the guide catheter 202 during a procedure. The physician can subsequently deliver a treating catheter over the guidewire 212, through a main lumen 204 of the guide catheter 202, and through a lumen 228 of the tube member 220 until the working portion of the treating catheter is located beyond the distal end 224 of the tube member. Through use of the tube member 220, the operating physician can shield the vasculature from abrasion or injury caused by advancement of the treating catheter toward the lesion 218. Additionally, the tube member 220 can provide added alignment support to the guide catheter 202 relative to the coronary ostium as the treating catheter is advanced.

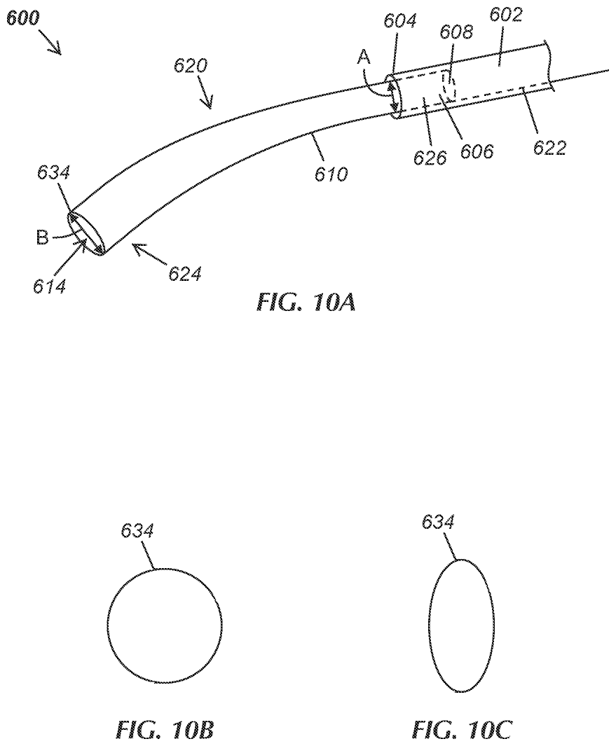

In general, the lumen 228, and hence the tube member 220 when expanded, can be sized and shaped to pass one or more interventional devices such as the guidewire and the treating catheter therethrough. The cross-sectional shape of the expanded lumen 228 can be similar to the cross-sectional shape of the guide catheter's main lumen 204. For instance, in some examples, the cross-sectional shape of the expanded lumen 228 can be generally uniform along its length. In other examples, the cross-sectional diameter may vary along the length of the tube member 220. According to embodiments of such examples, the distal end 224 of the tube member 220 may be narrower, e.g., tapered, relative to the proximal end 226, for instance. In additional examples, such as those described below in connection with FIG. 10A, the proximal end of a tube member may be narrower than the distal end. The length of each differently-sized portion of the tube member 220 in such embodiments can also vary, and in some examples, the distal end 224 of the tube member can be the longest. In examples that include differently sized proximal and distal ends, the difference in diameter between the proximal end 226 and the distal end 224 of the tube member may be from about 1 F to about 4 F, or anywhere in between.

The outer diameter of the tube member 220 when expanded can assume maximum cross-sectional dimensions that allow the tube member 220 to coaxially slide relative to the guide catheter 202. In other embodiments, the outer cross-sectional dimensions of the tube member 220 when expanded can be less than the allowable maximum. In varying embodiments, a diameter of the lumen 228 of the tube member 220 when expanded is not more than about one French size smaller than a diameter of the lumen 204 of the guide catheter 202. In one embodiment, the guide extension catheter 200 can be made in at least three sizes corresponding to the internal capacity of 8 F, 7 F, and 6 F guide catheters that are commonly used in interventional cardiology procedures. The difference in size between the outer diameter of the tube member 220 when expanded and the inner diameter of the guide catheter 202 may vary. For instance, the gap in cross-sectional diameter between the inner diameter of the guide catheter and the outer diameter of the tube member 220 when expanded may be less than and/or about 0.001 in., 0.002 in., 0.003 in., 0.004 in., or 0.005 in., or any distance therebetween. In specific embodiments, the cross-sectional diameter gap may range from about 0.002 to 0.003 in., or about 0.002 to 0.0035 in. For example, where a guide catheter has an inside diameter of 0.070 in. and the guide extension catheter has an expanded outside diameter of 0.068 in., the gap would be 0.002 in. The diameter gap between an outer diameter of the tube member 220 when expanded and the lumen 204 of the guide catheter 202 may also be generally continuous along a substantial portion of the length or a majority of the length of the tube member 220 in some embodiments, or the diameter gap may increase along one or more distal portions of the tube member 220.

The length of the tube member 220 can be substantially less than the length of the guide catheter 202; however, the tube member 220 can be designed with any length according to a desired application, such as about 6 to about 45 cm, about 10 to about 35 cm, about 14 to about 25 cm, or about 18 to about 20 cm.

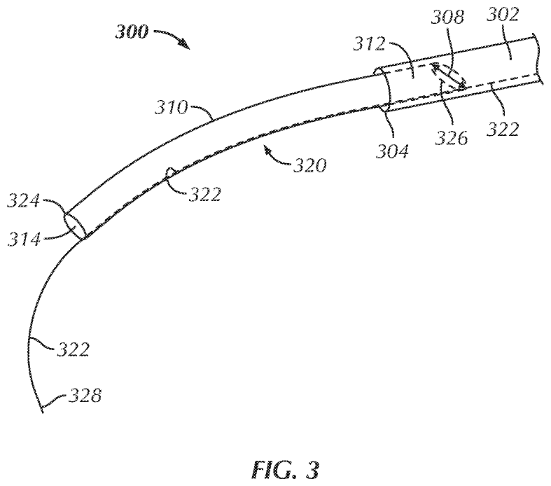

FIG. 3 illustrates an isometric view of a guide extension catheter 300 extending from a distal end 304 of a guide catheter 302. The guide extension catheter 300 includes a tube member 320 having a proximal end 326 and a distal end 324. FIG. 3 illustrates what is referred to herein as a “fixed wire” embodiment, in which a push member 322 is affixed to the tube member 320 and extends distally from of tube member 320. The push member 322 includes a distal tip 328.

The proximal end 326 of the tube member 320 can include a first reinforcement member 306, which may comprise an elongate tube or concave track configured to provide additional push strength during insertion of the guide extension catheter 300 within a vessel and/or configured to maintain a first lumen 308 of the guide extension catheter 300. In other embodiments, the first reinforcement member 306 may include a non-elongate tube, e.g., a tube or ring having an inner diameter greater than its length. The first reinforcement member 306 may be a complete or partial ring, for example, formed of a suitable polymer or metal to maintain a distended entry/exit point allowing free advancement and withdrawal of the guide extension catheter 300. In one embodiment, the first reinforcement member 306 can include polyether block amide having a durometer of 72, such as PEBAX 7233 available from Arkema. PEBAX 7233 has a Shore D hardness of 61, a tensile strength at yield of 3,770 psi, a tensile modulus of 74.0 ksi, and an elongate at yield of 18%. A lubricious layer of polytetrafluoroethylene (PTFE) can coat the inner surface of the PEBAX 7233.

The tube member 320 further includes a radially-collapsible tubular membrane 310 located distally of the first reinforcement member 306. A proximal end 312 of the tubular membrane 310 can be secured to a distal end 307 of the first reinforcement member 306, wherein the first reinforcement member 306 is configured to maintain patency of a lumen 314 of the tubular membrane 310. In one embodiment, the tubular membrane 310 can include a lubricious layer, a non-crosslinked binder layer, and a crosslinked heat shrink layer. The lubricious layer can include PTFE. The non-crosslinked binder layer can include polyether block amide having a durometer of 35, such as PEBAX 3533 available from Arkema. PEBAX 3533 has a Shore D hardness of 25, a tensile strength at break of 5,660 psi, and a tensile modulus of 2.61-2.76 ksi. The crosslinked heat shrink layer can include polyether block amide having a durometer of 55, such as PEBAX 5533 available from Arkema. PEBAX 5533 has a Shore D hardness of 50, a tensile strength at yield of 1,740 psi, and a tensile modulus of 23.9-24.7 ksi.

In various embodiments, the tubular membrane 310 can have a column strength, a radial strength, and a bend stiffness significantly less than that of the first reinforcement member 306.

The column strength, the radial strength, and the bend stiffness of the tubular membrane 310 may be non-effective, i.e., the radially-collapsible, tubular membrane 310 can have no effective column strength, no effective radial strength, and no effective bend stiffness. In other words, any radial force, column force, or bend force can cause the tubular membrane 310 to deflect, collapse, and/or bend, and the tubular membrane 310 will provide no effective resistance to a radial, column, or bend force.

The tubular membrane 310 can have a tensile strength sufficient to prevent tearing of its walls during advancement and withdrawal of an interventional device. For instance, during advancement of an interventional device through the tubular membrane 310, the interventional device may provide a force which urges the walls of the tubular membrane 310 radially outward. The tubular membrane 310 may have a tensile strength sufficient to withstand the radially outward force of the interventional device such that the interventional device does not tear or otherwise damage the walls of the tubular membrane 310.

The tubular membrane 310 may be durably lubricious on one or both of its inner surface or its outer surface via a hydrophobic, silicone, or polymer coating. The lubricious inner surface of the tubular membrane 310 may be configured to reduce friction between the tubular membrane 310 and the interventional device during insertion and/or withdrawal of the device. The lubricious outer surface of the tubular membrane 310 may be configured to reduce friction between the tubular membrane 310 and the guide catheter 302 during insertion and/or withdrawal of the guide extension catheter 300 from the guide catheter 302.

FIG. 3 illustrates the lumen 314 of the tubular membrane 310 in a fully-open/fully-expanded state. In the fully-open state, the lumen 314 of the tubular membrane 310 has a first inner diameter, a first outer diameter, and a first cross-sectional area. The tubular membrane 310 includes a wall thickness of 0.00075-0.004 in., and an outer diameter to wall thickness ratio of 10:1 to 200:1, or optionally, an outer diameter to wall thickness ratio of 10:1 to 50:1, such as 18:1, 24:1, 30:1, 36:1, 42:1 and ranges therebetween. The thin-walled nature of the tubular membrane 310, and therefore larger cross-sectional area for a given compatible guide catheter size, allows interventionalists to perform a wider array of challenging procedures in today's world of complex intervention via customary femoral access or new-age radial access.

In some embodiments, the lumen 314 of the tubular membrane 310 may be freely collapsible, i.e., the tubular membrane 310 cannot support a first cross-sectional area without exterior support (e.g., the first reinforcement member 306 supporting the patency of the lumen 314) or without interior support (e.g., the interventional device inserted through the tubular membrane opens the lumen via an interior force). Thus, if the tubular membrane 314 is not supported by an additional component or feature, the lumen 314 will “collapse”, meaning the cross-sectional area of the lumen 314 will be less than the first cross-sectional area (i.e., the cross-sectional area in the fully-open state illustrated in FIG. 3).

In the collapsed state, the tubular membrane 310 may exhibit no effective tensile strength, i.e., the tubular membrane 310 will provide no effective resistance of a tensile force when the cross-sectional area of the lumen 314 is less than the cross-sectional area in the fully open state. However, in the fully-open state, the tubular membrane 310 may exhibit a tensile strength sufficient to prevent tearing of the walls of the tubular membrane 310 and sufficient to prevent micro and/or macro injury to the vessel wall during insertion of an interventional device. In other words, when the cross-sectional area of the tubular membrane 310 is expanded to the fully-open state, the tubular membrane 310 will provide resistance to any radially-outward tensile force. In various embodiments, the tensile strength of the tubular membrane may exceed about 2.25 lbs.

In the embodiment illustrated in FIG. 3, the push member 322 extends distally from the distal end 324 of the tube member 320. The push member 322 may comprise a stainless steel or nitinol core wire and/or a solid core wire wrapped in a smaller wire coil. A distal tip 328 of the push member 322 may include an atraumatic guidewire-like distal tip. In some embodiments, the atraumatic guidewire-like distal tip includes a tapered core surrounded by a coil, and in some embodiments, the atraumatic guidewire-like distal tip includes a steerable tip. The push member 322 may serve as a backbone to the tubular membrane 310, i.e., the push member 322 may provide column strength and/or bend stiffness to facilitate advancement and withdrawal of the guide extension catheter 300. In some embodiments, a proximal portion of the push member 322 may include or be surrounded by a removable support member as described in commonly-owned U.S. Pat. No. 10,953,197, which is hereby incorporated by reference in its entirety.

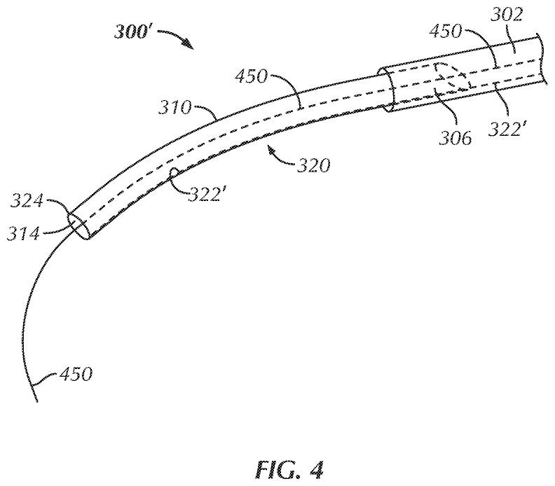

FIG. 4 illustrates an isometric view of a guide extension catheter 300′ and a guidewire 450, according to some embodiments. The guide extension catheter 300′ is referred to herein as a “rapid exchange” embodiment wherein the push member 322′ terminates at or before the distal end 324 of the tube member 320, and the guidewire 450 extends distally beyond the distal end 324 to guide the guide extension catheter 300′ and/or interventional device. The guidewire 450 may be slidable relative to the tube member 320, or rather, the tube member 320 may be slid over the guidewire 450 and advanced into a position adjacent to the target area.

The guidewire's 450 slidable nature relative to the guide extension catheter 300′ enables a rapid exchange of interventional devices. For instance, an interventional device such as a balloon dilator (not shown) may be slidably advanced on the guidewire 450. The balloon dilator may be advanced through the guide catheter 302 and the guide extension catheter 300′, and advanced distally from the distal end 324 of the tube member 320 to target tissue. A treatment may be provided at the target tissue (e.g., the balloon dilator can be inflated to open a vessel lesion), and the balloon dilator may be slidably retracted on the guidewire 450 from the target tissue and through the guide extension catheter 300′ and the guide catheter 302. The balloon dilator may be withdrawn from the patient and removed from the guidewire 450. A second interventional device such as a stent may then be slidably received on the guidewire 450 and advanced through the guide catheter 302, through the guide extension catheter 300′, and to the target tissue located distally of the distal end 324 of the tube member 320. A second treatment may be provided at the target tissue (e.g., the stent can be deployed). Thus, the guidewire 450 enables a rapid exchange of interventional devices.

In some embodiments, the push member 322′ can terminate in a second reinforcement member 334 of the guide extension catheter 300′. The push member 322′ may be directly secured to the second reinforcement member 334, wherein the push member 322′ is configured to provide additional push strength during insertion of the guide extension catheter 300 within a vessel and the second reinforcement member 334 is configured to maintain the lumen 314 of the tubular membrane 310. The second reinforcement member 334 can be sufficiently flexible and deflectable along an axis of the guide extension catheter 300′ such that it conforms to create a reduced crossing profile when resistance to advancement is encountered. The second reinforcement member 334 can be stowed and deployed outside of the guide catheter. For example, a hollow guidewire or push member 322′ can allow for a second reinforcement member 334 in the form of a retractable distal reinforcing ring.

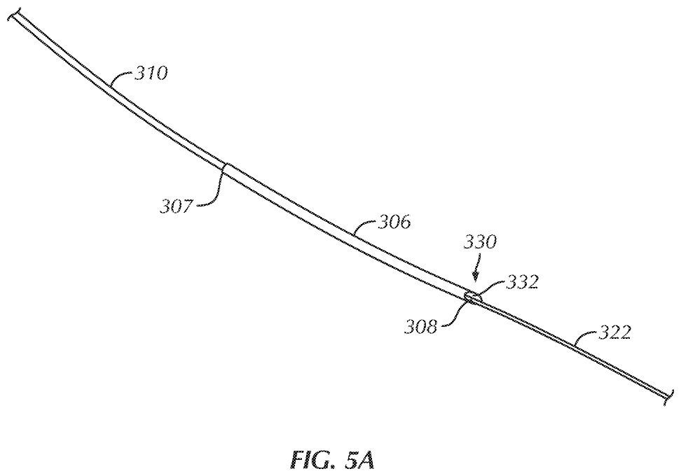

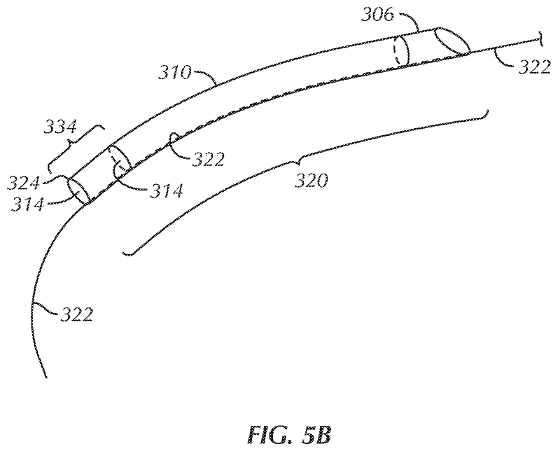

FIG. 5A illustrates an isometric view of the guide extension catheter 300 showing a first reinforcement member 306. A proximal end 330 of the first reinforcement member 306 may include a concave opening 332 leading into the first lumen 308. The concave opening 332 may be configured to ease the insertion of a guidewire (not shown) or an interventional device (not shown) into the first lumen 308 of the first reinforcement member 306. For instance, the concave opening 332 may provide a larger area to receive an interventional device into the tube member than an area associated with an opening oriented perpendicular to the longitudinal axis of the tube member. In some embodiments, the first reinforcement member 306 may include an extended concave track defining a half-pipe feature configured to help guide an interventional device into the first lumen 308 of the first reinforcement member 306. Exemplary embodiments of half-pipes, concave tracks, and other first reinforcement members and features are described in commonly-owned U.S. Pat. Pub. No. 2019/0247619, which is hereby incorporated by reference in its entirety.

The first reinforcement member 306 can have a length l defined between the proximal end 330 and the distal end 307. The first reinforcement member 306 can have an inner diameter dᵢ (also referred to as a first lumen diameter), an outer diameter dₒ, and a wall thickness. In some embodiments, the length l of the first reinforcement member may be as small as dᵢ/10.

In some embodiments, the first reinforcement member 306 can be formed from an inner polymer layer, an outer polymer layer, and/or a reinforcement layer (e.g., braid or coil) disposed between or adjacent to the polymer layers. According to such examples, the inner polymer layer can be composed of, or coated with, silicone, PTFE or another lubricious material to provide a slippery surface for received interventional devices. The outer polymer layer can include one or more materials, such as polyurethane, polyethylene, polyolefin, or polyether block amide of sequentially diminishing durometers along the tube member's length, and it can be coated with a friction-reducing material (e.g., a hydrophilic material) to facilitate insertion and trackability through vasculature and a guide catheter. The reinforcing braid or coil, in embodiments featuring a braid or coil, can be formed of stainless steel or a platinum alloy, for example, and can extend between the polymer layers along at least a portion of the tube member's length.

In some embodiments, the push member 322 may include one or more depth markers, which may be positioned at predetermined lengths relative to a distal end of the tube member 320. One or more radiopaque marker bands may be positioned on the tube member 320. The marker bands can be composed of tungsten, platinum or an alloy thereof and can have a metallic band structure. Alternatively, for space conservation reasons, the marker bands can be formed by impregnating portions of the tube member 320 with a radiopaque filler material, such as barium sulfate, bismuth trioxide, bismuth carbonate, powdered tungsten, powdered tantalum or the like.

FIG. 5B illustrates the tube member 320 comprising a second reinforcement member 334 disposed at the distal end 324 of the tube member 320. The second reinforcement member 334 may be configured to maintain patency of the lumen 314 at the distal end 324 of the tubular membrane 310 such that the interventional device may be withdrawn from the lesion area and back into the lumen 314 of the guide extension catheter 300. In some embodiments, the second reinforcement member 334 may be configured to selectively open and/or close the distal end 324 of the tubular membrane 310.

In some embodiments, the second reinforcement member may include a small diameter snare-like device that emerges from a hollow push member 322, such as the snare device described in commonly-owned U.S. Pat. Pub. No. 2005/0234474, which is hereby incorporated by reference in its entirety.

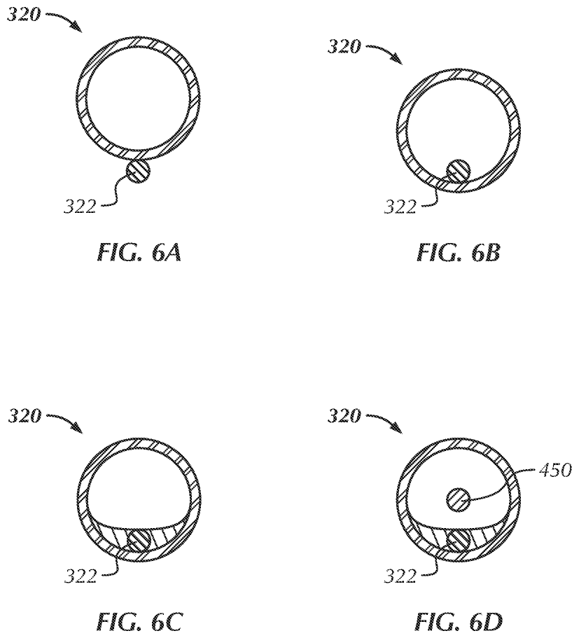

As shown in FIGS. 5A-5B, the push member 322 may be affixed to an inner surface of the tube member 320 (see also FIG. 6B). In other embodiments, e.g., FIGS. 6A and 6C-6D, the push member 332 may be affixed to an outer surface of the tube member 320 (see, e.g., FIG. 6A) or integrated within a wall of the tube member 320 such as sandwiched between an inner polymer layer and an outer polymer layer (see, e.g., FIGS. 6C-6D). The location of the push member 322 may be configured to maximize a first lumen area, i.e., to maximize the cross-sectional area within the lumen of the first reinforcement member 306 to allow for a larger interventional device delivery through the guide catheter. In other embodiments, the location of the push member 322 may be configured to minimize an outer diameter, i.e., to allow delivery of the guide extension catheter 300 through the guide catheter 302. In further embodiments, the location of the push member 322 may be configured to control flexibility, steerability, and/or deflection of the guide extension catheter 300.

As illustrated in FIGS. 6A-6D, the push member 322 can define a circular cross-section along a portion of its length. However, the cross-sectional shape and dimensions of the push member 322 may vary. For example, the push member 322 can comprise an arcuate or flat, sheet-like cross-sectional shape, and rectangular, irregular, oval, oblong cross-sectional shapes are also within the scope of this disclosure. Exemplary embodiments of push members are described in commonly-owned U.S. Pat. No. 10,751,514, which is hereby incorporated by reference in its entirety. The stiffness of the push member 322 may be uniform, or substantially uniform, along its length, or may define areas having variable stiffness along its length. For example, the push member 322 may be more flexible near its distal end than its proximal end. The push member 322 may include sufficient rigidity to avoid helical twisting of the guide extension catheter 300 during use.

FIG. 6D illustrates a cross-sectional view of the tube member 320 having the push member 322 integrated within a wall and the guidewire 450 extending therethrough. The guidewire 450 may be slidable relative to the tube member 320, i.e., the guidewire 450 may slide in a longitudinal direction relative to the longitudinal axis of the tube member 320. In some embodiments, the guidewire 450 may also be able to travel laterally and vertically within the tube member (e.g., the guidewire 450 can move around the inner circumference, or near the center point, of the tube member 320).

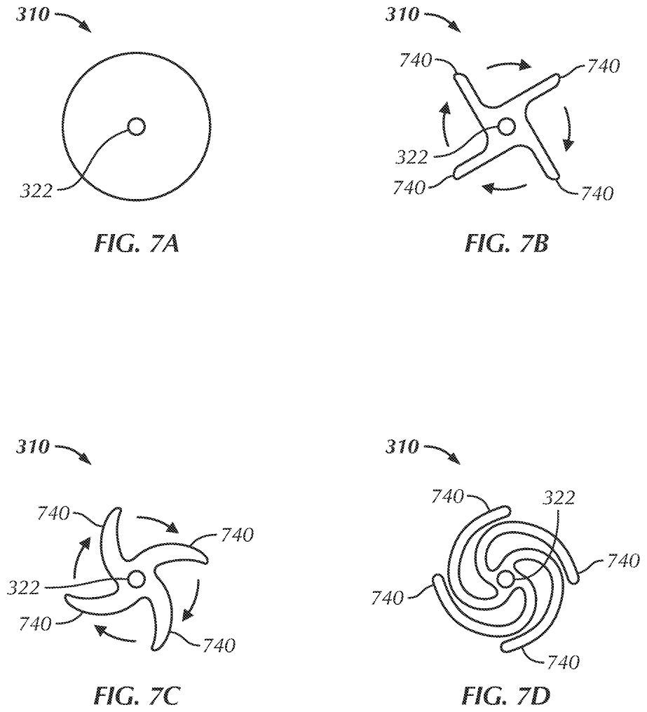

As stated above, the tubular membrane 310 may be freely collapsible and/or exhibit no effective column strength, radial strength, or bend stiffness. Thus, the tubular membrane 310 may be loosely draped over the push member 322, 322′ and/or guidewire 450 prior to insertion into a patient. In some embodiments, the tubular membrane 310 may be folded around the push member 322, 322′ to reduce cross-sectional area and/or to reduce friction forces during insertion. FIGS. 7A-7D illustrate cross-sectional views of an exemplary folding of the tubular membrane 310 of the guide extension catheter 300. The push member 322 is disposed near the center of the tubular membrane, however, in other embodiments, the push member 322 may be off-center from the tubular membrane, secured to an inner side wall, an outer side wall, or integrated within a wall of the tubular membrane 310 (e.g., see FIGS. 6A-6D).

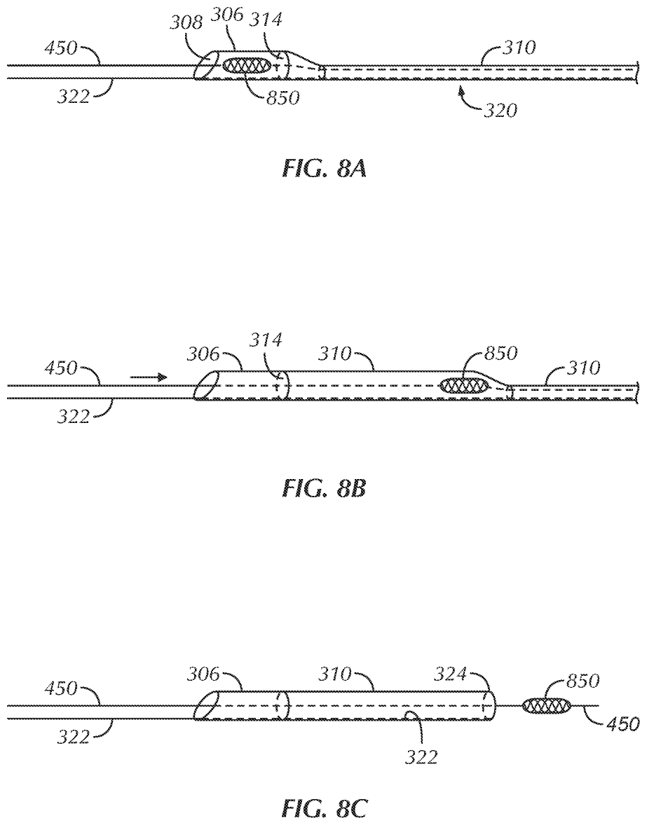

In the embodiment shown in FIGS. 7A-7D, the tubular membrane 310 is shaped to include one or more fold flaps 740, in this case, four fold flaps 740. The one or more fold flaps 740 are rotated around the push member 322 to reduce the cross-sectional profile of the guide extension catheter 300. In some embodiments, the one or more fold flaps 740 may include a variety of different fold geometries and sizes. The fold flaps 740 may be configured to open, or unfold, as an interventional device is inserted through the tubular membrane 310. For instance, as an interventional device is inserted through the folded tubular membrane 310, the interventional device may provide a radially outward force upon the tubular membrane 310 to urge the walls radially outward, and thus, the fold flaps 740 are urged outward and unfold. An illustration of the unfolding and/or opening of the tubular membrane is illustrated in FIGS. 8A-8C.

FIGS. 8A-8C illustrate an exemplary progression of an interventional device 850 being inserted through the guide extension catheter 300. In some embodiments, the guidewire 450 may be positioned within a patient before the guide extension catheter 300 and the interventional device 850 are inserted, and the guidewire 450 may be configured to guide the guide extension catheter 300 into the correct position. In this way, the guide extension catheter 300 may slide over the guidewire 450 and follow the guidewire 450 to a position adjacent to a target tissue area. The guide extension catheter 300 may also be guided or steered into position via the guide catheter 302 (not illustrated in FIGS. 8A-8C). The guide extension catheter 300 may be slidably advanced toward a target tissue area via applying a force to the push member 322. In some embodiments, a delivery assist device (e.g., the device described in commonly-owned U.S. Pat. No. 8,048,032, which is hereby incorporated by reference in its entirety) may be used to help open the lumen 314 of the tubular membrane 310.

The interventional device 850 may be slidably secured to the guidewire 450 to move longitudinally down the length of the guidewire 450. As illustrated in FIG. 8A, the interventional device enters the first reinforcement member 306 of the tube member 320. The first reinforcement member may be configured to maintain patency of a first lumen 308 of the tube member 320, such that the interventional device 850 can be easily inserted into the tube member 320. The first reinforcement member 306 may be configured to maintain patency of the lumen 314 of the tubular membrane 310 at a proximal end 312 of the tubular membrane 310. In some embodiments, the tubular membrane 310 may be folded along a substantial portion of its length prior to insertion of the interventional device 850 (see, e.g., FIGS. 7A-7D). In some embodiments, the tubular membrane 310 may be freely collapsed, i.e., the cross-sectional area of the lumen 314 is less than the cross-sectional area in the fully open state (see, e.g., FIG. 3).

As illustrated in FIG. 8B, the interventional device 850 may advance distally of the first reinforcement portion 306 and enter the tubular membrane 310. The lumen 314 of the tubular membrane 310 may open as the interventional device 850 is advanced through the tubular membrane 310. For instance, in embodiments where the tubular membrane 310 was folded along a substantial portion of its length (see, e.g., FIGS. 7A-7D), advancement of the interventional device 850 may unfold the tubular membrane 310 and push the walls of the tubular membrane 310 radially outward, increasing the cross-sectional area of the lumen 314. In embodiments where the tubular membrane 310 was freely collapsed prior to insertion of the interventional device, the lumen 314 of the tubular membrane 310 may be urged open as the interventional device 850 is advanced through the tubular membrane 310.

As illustrated in FIG. 8C, the interventional device may advance distally of the distal end 324 of the tube member 320 to deliver a treatment at or near a target tissue location. In some embodiments, the tube member 320 may include a second reinforcement member 334 disposed at the distal end 324 of the tube member 320. The second reinforcement member 334 (not shown in FIGS. 8A-8C) may be configured to maintain the lumen 314 of the tubular membrane 310 at the distal end 324, such that the retraction of the interventional device 850 from the target tissue area into the tube member 320 is unimpeded. For instance, if the distal end 324 of the tube member 320 was collapsed or folded, the lumen 314 may be smaller than the cross-sectional size of the interventional device 850, resulting in twisting, kinking, or folding of the walls of the tubular membrane 310 upon the retraction of the interventional device 850. The second reinforcement member 334 may comprise a tube or ring having a column strength, radial strength, and/or bend stiffness greater than the respective column strength, radial strength, and/or bend stiffness of the tubular membrane 310.

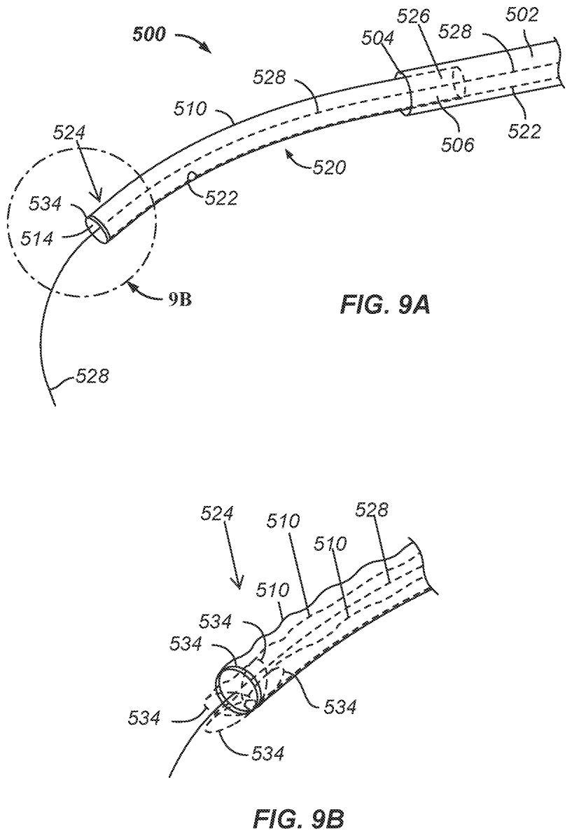

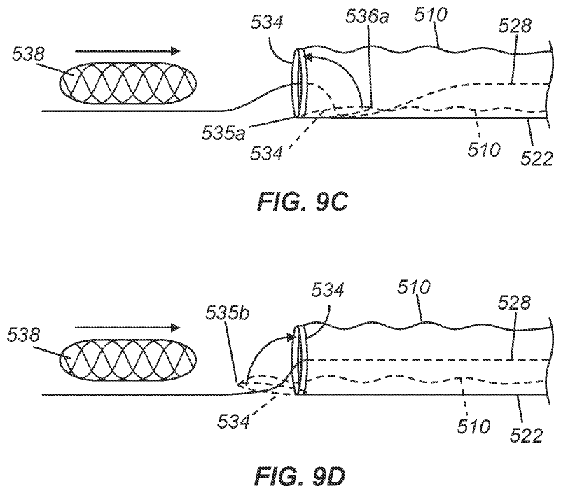

FIG. 9A provides an isometric view of a guide extension catheter featuring a distal reinforcement member configured to pivot, lean, tilt, or otherwise undergo a change in angular position upon contacting a proximally retracting interventional device in a manner that opens the passageway for the device defined by the lumen of the tube member. Shown extending from a distal end 504 of a guide catheter 502, the guide extension catheter 500 includes a tube member 520 having a distal end 524, a proximal end 526, and a radially-collapsible tubular membrane 510 with a push member 522 affixed thereto. A guidewire 528 is extended through and distally beyond the distal end 524 of the tube member 520, through a lumen 514 of the tubular membrane 510, which is depicted in a fully-open/fully-expanded state. The tubular membrane 510 may be freely collapsible and/or foldable, consistent with embodiments of the tubular membrane described herein.

The proximal end 526 of the tube member 520 may include a first reinforcement member 506, which may comprise a substantially circular component, e.g., a ring or coil. Embodiments of the reinforcement member 506 may also include or comprise features similar or identical to reinforcement member 306, such as an elongate tube, concave track, a non-elongate tube (e.g., partial or complete ring or coil), or a combination thereof. Embodiments of the first reinforcement member 506 may comprise various materials, non-limiting examples of which may include one or more metals, plastics, or combinations thereof.

As further shown in FIG. 9A, the distal end 524 of the tube member 520 may include a second, distal reinforcement member 534 comprising a resilient support ring or coil member (hereinafter referred to as “coil member 534”) attached to the distal end of the tubular membrane 510 and configured to facilitate entry of one or more interventional devices, e.g., treatment catheters, into the tube member 520 during proximal retraction of such devices from a target site within the vasculature of a subject. The coil member 534 may comprise a flexible, deformable, shape-memory material and/or resilient configuration that is biased toward a resting, unconstrained configuration in which the coil member 534 is substantially perpendicular or otherwise orthogonal to a longitudinal axis of the push member 522, as shown in FIG. 9A. The unconstrained configuration of the coil member 534 may establish, expand, and/or maintain a patency of at least a distal portion of the lumen 514 of the tubular membrane 510. In its resting state, not positioned within a guide catheter or confined within an operational space within the vasculature of a subject, the coil member 534 may thus define a distal opening into the tube member 520 oriented and sized to accommodate unobstructed entry and passage of one or more interventional devices therethrough.

When confined within a blood vessel during an interventional procedure, the coil member 534 may tilt, bend, or otherwise lean in a proximal or distal direction, as represented by the dashed outlines of the coil member 534 shown in FIG. 9B. In this configuration, and when no interventional device is positioned within the tubular membrane 510, the membrane 510 may be in its collapsed state, loosely draped or folded over the push member 522, which may, in some examples, not be especially conducive to retrograde entry of an interventional device from beyond its distal end due to a potential risk of the membrane interfering with the path of the interventional device. To facilitate entry of an interventional device 538 being retracted proximally toward the distal end 524 of the tube member 520 (in the direction of the horizontal arrows in FIGS. 9C and 9D), contact of the interventional device 538 with the leading edge or distal-most portion 535a, 535b of the leaning coil member 534 may cause the coil member 534 to swing or pivot toward its unconstrained configuration substantially perpendicular or orthogonal to the longitudinal axis of the push member 522, thereby transitioning the distal end of the lumen 514 of the tubular membrane 510 to its expanded, non-collapsed state and opening a clear passageway through the opening defined by the coil member 534 and into the tube member 520 through which the interventional device may be retracted proximally. Accordingly, the angulation of the coil member 534 with respect to the longitudinal axis of the push member 522 may change in response to forces applied by an interventional device. Due to its material composition and configuration, the angulation and/or cross-sectional form of the coil member 534 may also change in response to variations in the vasculature of a subject, e.g., widening and narrowing of tortuous blood vessels.

In embodiments featuring a coil member 534 that leans proximally when confined within a guide catheter 502 and/or blood vessel (see dashed coil member 534 in FIG. 9C), contact between the distal-most portion 535a of the coil member 534 and a proximally retracting interventional device 538 may cause the opposite, “free” end 536a of the coil member 534 to pivot or swing in the direction of the curved arrow shown in FIG. 9C, toward its unconstrained resting configuration, bringing the attached distal end of the tubular membrane 510 therewith. When leaning distally when confined within a guide catheter 502 and/or blood vessel (see dashed coil member 534 in FIG. 9D), contact between the distal-most portion 535b of the coil member 534 (constituting the “free” end in this configuration), and a proximally retracting interventional device 538 may cause the distal-most portion 535b of the coil member 534 to pivot or swing in the direction of the curved arrow shown in FIG. 9D, toward its unconstrained resting configuration, again bringing the attached distal end of the tubular membrane 510 therewith. Accordingly, proximal retraction of an interventional device may cause the coil member 534 to return to, or near, its resting, unconstrained state in which the cross-sectional area of the lumen 514 of at least a distal portion of the tubular member 510 is increased or maximized, and interference with a proximally retracting interventional device is reduced together with the likelihood of any associated damage to the tubular membrane 510. The angulation of the coil member 534 may thus be directly responsive to the position and movement of the interventional device 538, along with the dimensions and curvature of the surrounding vessel wall.

Embodiments of the coil member 534 may be integrally formed with the push member 522, such that the coil member 534 may be continuous with the push member 522 and, in some examples, may define the distal end of the push member 522. According to such embodiments, the push member 522 may comprise an elongate push rod or body having a longitudinal axis that aligns substantially with the longitudinal axis of the tube member 520 and guide catheter 502, and a distal portion that coils, curls, or otherwise curves away from its longitudinal axis. In other embodiments, the coil member 534 may comprise a distinct component, portion, or segment affixed, coupled, or otherwise attached to the distal end of the push member 522, for instance via welding.

FIG. 10A provides an isometric view of a guide extension catheter configured to receive proximally retracting interventional devices via a tube member that varies in cross-sectional area along its length. As shown, the guide extension catheter 600 includes a tube member 620 having a distal end 624, a proximal end 626, and a radially-collapsible tubular membrane 610 with a push member 622 affixed thereto. A guidewire may be extended through and distally beyond the distal end 624 of the tube member 620, through a lumen 614 of the tubular membrane 610, which is depicted in a fully-open/fully-expanded state. In the example shown, the radially-collapsible tubular membrane 610 is tapered such that the cross-sectional area of the distal end 624 of the tube member 620 is greater than the cross-sectional area of the proximal end 626 of the tube member 620 in the expanded state. The proximal end 626 of the tube member 620 may include a first reinforcement member 606, which may be similar or substantially identical to reinforcement member 306 and/or 506.

A distal portion 624 of the tube member 620 may include or be defined by a distal reinforcement member 634 attached to the distal end of the tubular membrane 610. The distal reinforcement member 634 may comprise a flexible, shape-memory material and/or resilient configuration configured to flex radially outward, into an expanded state, when advanced distally beyond a distal end 604 of a guide catheter 602. Embodiments of the distal reinforcement member 634 may comprise a resilient circular element, such as a ring or coil, which may be deformable and configured to undergo angular adjustments relative to the longitudinal axis of the push member 622, like coil member 534. In the expanded configuration, the tube member 620 may assume a conical or funnel shape in which the diameter, and thus cross-sectional area, of the tube member 620 is the largest at its distal end 624. Radial expansion or flexing of the distal reinforcement member 634, and thus concomitant widening of at least a distal portion of the lumen 614 of the tubular membrane 610 attached thereto, may occur automatically in spring-like fashion as it is released from the guide catheter 602, thereby opening or expanding the cross-sectional space available for entry and smooth passage of interventional devices through the distal end 624, for example during proximal retraction of such devices during or upon completion of an interventional procedure.

Together with the self-expandable, flexible configuration of the distal reinforcement member 634, this distal expansion of the tube member 620 may be made possible by the narrow cross-sectional wall thickness and small cross-sectional space occupied by the radially-collapsible tubular membrane 610 in its collapsed state. The compactness of the tubular membrane 610 in its collapsed configuration may configure the tube member 620 for advancement through a guide catheter 602 having a smaller inner diameter relative to at least a portion of the inner and/or outer diameter of the tube member 620 in its expanded state. The cross-sectional diameter of the tube member 620 along at least a distal portion thereof, in its expanded state, may therefore not be limited by the inner diameter of the guide catheter 602. As a result, the guide extension catheter 600 may be compatible with a wider variety of interventional devices than preexisting guide extension devices, and may accommodate the simultaneous passage of more than one interventional device during a given procedure, thereby enhancing the ease with which a broad array of interventional procedures are performed.

The cross-sectional dimensions of the tube member 620 along its length in the expanded state may vary. In some examples, after its advancement beyond the distal end 604 of a guide catheter 602, at least a distal portion of the tube member 620, e.g., having diameter B, may expand until its outer perimeter abuts the inner surface of a surrounding vessel wall. In such embodiments, at least a portion of the tube member 620 in its expanded state may have an outer diameter approximately equal to or greater than the inner diameter of the guide catheter 602. Variously sized guide catheters 602 can be used, e.g., 6 F, 7 F, or 8 F guide catheters, and the diameter variation between the proximal end 626 and distal end 624 of the tube member 620 may vary by about 1 F to about 4 F, or anywhere in between. The cross-sectional gap in size between the inner diameter of the guide catheter 602 and the outer diameter of the tube member 620 at or along at least a portion of its length in its expanded state may also vary. For instance, the gap between the inner diameter of the guide catheter 602 and the larger outer diameter of at least a portion of the tube member 620 when extended beyond the distal end 604 of the guide catheter 602 may be less than, greater than, and/or about 0.001 in., 0.002 in., 0.003 in., 0.004 in., 0.005 in. 0.006 in. 0.007 in. 0.008 in. 0.009 in. 0.010 in., 0.011 in., 0.012 in., 0.013 in., 0.014 in., 0.015 in., or any distance therebetween.

The cross-sectional diameter, and thus area, of the tube member 620 extended beyond the distal end 604 of the guide catheter 602 may increase in a smooth, gradient fashion such that the resulting conical configuration defines a constant or substantially constant slope. In other embodiments, the expanded tube member 620 may define one or more discrete segments or steps defining different slopes. Accordingly, the cross-sectional area of the expanded tube member 620 may increase in the distal direction in a smooth or step-like fashion.

The proximal end 626 of the tube member 620 remaining nestled within the guide catheter 602 may have the maximum cross-sectional dimensions that allow the tube member 620 to coaxially slide relative to the guide catheter 602. In other embodiments, the outer diameter of at least a proximal portion of the tube member 620 may be less than the allowable maximum. In various embodiments, a diameter of a first lumen 608 of the tube member 620 positioned within the guide catheter 602, e.g., diameter A, may not be more than about one French size smaller than an inner diameter of the lumen of the guide catheter 602. In one embodiment, the guide extension catheter 600 may be made in at least three sizes corresponding to the internal capacity of 8 F, 7 F, and 6 F guide catheters that are commonly used in interventional cardiology procedures. The difference in size between the outer diameter of at least a proximal portion 626 of the tube member 620 and the inner diameter of the guide catheter 602 may vary. For instance, the gap in cross-sectional diameter between the inner diameter of the guide catheter 602 and the maximum outer diameter of at least a proximal portion 626 of the tube member 620, for example at the proximal reinforcement member 606, may be less than and/or about 0.001 in., 0.002 in., 0.003 in., 0.004 in., or 0.005 in., or any distance therebetween. In specific embodiments, the cross-sectional diameter gap may range from about 0.002 to 0.003 in., or about 0.002 to 0.0035 in. The diameter gap between a maximum outer diameter of at least a proximal portion of the tube member 620 and the lumen of the guide catheter 602 may also be generally continuous along a substantial portion of the length or a majority of the length of the tube member 620 in some embodiments, or the diameter gap may increase along one or more portions of the tube member 620.

FIGS. 10B and 10C provide en face views of the distal reinforcement member 634 in different configurations, demonstrating its flexibility, resiliency, and deformable properties. FIG. 10B shows the distal reinforcement member 634 in a substantially circular configuration, which may be its resting, unconstrained configuration unbound by a narrow blood vessel or guide catheter. This configuration may flex into a more oblong, oval configuration, as shown in FIG. 10C, in response to externally applied compression, constriction within a guide catheter, and/or anatomical variations, including blood vessel tortuosity and/or narrowing. The shape and configuration of the reinforcement member 634 may substantially conform to the inner surface of any surrounding structures, such that its configuration may vary widely in different environments. Embodiments of the coil member 534 shown in FIGS. 9A-9D may be configured similarly or identically in whole or in part, such that the distal reinforcement member 634 and coil member 534 may be flexible not only in their angulation with respect to the longitudinal axis of a push member, but also in their cross-sectional form. These deformable, deflectable, and/or flexible properties may significantly enhance the versatility of the guide extension catheter 600 (and guide extension catheter 500, for example). For instance, the distal reinforcement member 634 (and coil member 534, for example) may become narrower by ellipsoid deformation in order to pass through arterial segments that are relatively stenotic. Ellipsoid deformation of the distal reinforcement member 634 may also be instrumental for the tube member 620 to pass through a guide catheter having a smaller cross-sectional area. Accordingly, radial expansion of the distal reinforcement member 634 may refer to or coincide with, in whole or in part, a change in its cross-sectional form that occurs when it exits the distal end 604 of the guide catheter 602, such that the cross-sectional area of the distal reinforcement member 634 may increase even though its circumference remains the same. Within the guide catheter 602, the distal reinforcement member 634 may be deformed into a variety of cross-sectional configurations, for example including the oblong conformation shown in FIG. 10C. Upon exiting the guide catheter 602, the distal reinforcement member 634 may assume a more ring-like, circular cross-sectional configuration, such as that depicted in FIG. 10B. Its cross-sectional configuration may then continue to adjust in response to anatomical variations encountered during its advancement through a subject's vasculature, for example such that it may return, temporarily, to an approximately oblong or irregular cross-sectional shape when passing through a tightly curving or narrow blood vessel.

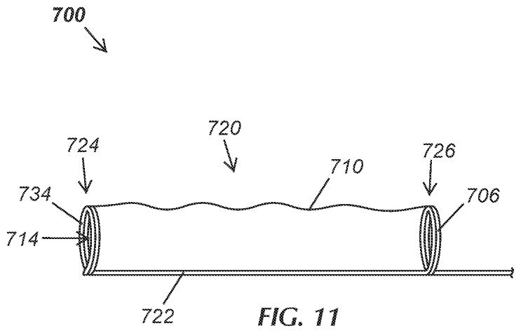

FIG. 11 shows a guide extension catheter featuring a tube member comprising a radially-collapsible, tubular membrane flanked by two inflatable reinforcement members configured to enable selective, controlled expansion of the tube member. In particular, the guide extension catheter 700 includes a tube member 720 having a distal end 724, a proximal end 726, and a radially-collapsible tubular membrane 710 affixed to a hollow push member 722. A first reinforcement member 706 comprising one or more inflatable, helical coils is included at the proximal end 726 of the tube member 720, and a second reinforcement member 734 comprising one or more inflatable, helical coils is included at the distal end 724 of the tube member 720. The lumen of each coiled structure, which may comprise a flexible, inflatable balloon arranged in a helical configuration, may be continuous with that of the hollow push member 722, such that when desired, the reinforcement members may be inflated via injection of inflation fluid into a proximal end of the hollow push member 722. Inflation of the reinforcement members 706, 734 transitions the tubular membrane 710, and thus tube member 720 as a whole, from a collapsed configuration to an expanded configuration, the latter depicted in FIG. 11, to accommodate entry and passage of one or more interventional devices therethrough.

The illustrated example includes two inflatable reinforcement members 706, 734, but additional embodiments may feature only one, for example disposed at a distal or proximal end of the tube member 720. Embodiments having only one inflatable reinforcement member may include an additional, non-inflatable reinforcement member comprising one or more of the features described above, for example in connection with reinforcement members 306, 334, 506, 534, 606, and/or 634. For instance, a guide extension catheter may include a proximal reinforcement member comprising one or more inflatable coils, and a distal reinforcement member comprising a resilient ring or coil, such as coil member 534 or distal reinforcement member 634.

In operation, one or both reinforcement members 706, 734 of the guide extension catheter 700 may be inflated to establish and/or maintain a patency of the lumen 714 of the radially-collapsible tubular membrane 710, thereby facilitating the entry and passage of one or more interventional devices distally or proximally therethrough. One or more cycles of inflation and subsequent deflation of the reinforcement member(s) may be performed over the course of a given procedure to accommodate the exchange of various interventional devices.

The number of complete helical coils included in each reinforcement member 706, 734 may vary. Embodiments may feature one or more helical coils, for example about two coils, three coils, four coils, five coils, or more. In embodiments featuring two reinforcement members, the number of inflatable coils included in each reinforcement member may be the same or different. For example, a proximal reinforcement member may feature two inflatable coils, while a distal reinforcement member may include only one.

The coils of the reinforcement members 706, 734 may be formed according to a variety of methods, one of which may involve winding an inflatable tube in a helical manner around a central axis into a series of windings subsequently stacked against and bonded to each other, for example as described in U.S. Pat. Nos. 10,946,177, 10,159,821, and 9,968,763, the contents of which are hereby incorporated by reference in their entireties.

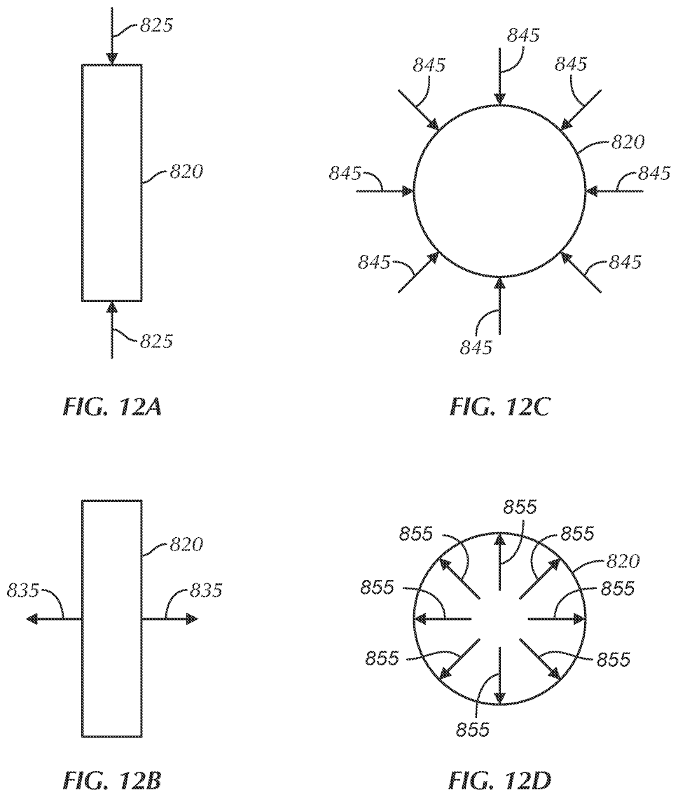

FIGS. 12A-12D illustrate examples of column strength, bend stiffness, radial strength, and tensile strength, respectively. FIG. 12A illustrates a diagrammatic top view of a tube member 820, with arrows 825 indicating a column force. Thus, the term “column strength” refers to the structural resistance to the column force(s) 825. FIG. 12B illustrates a diagrammatic top view of the tube member 820, with arrows 835 indicating a bend force. Thus, the term “bend stiffness” (or bend strength) refers to the structural resistance to the bend force(s) 835. FIG. 12C illustrates a diagrammatic cross-sectional view of the tube member 820, with arrows 845 indicating a radial compressive force. Thus, the term “radial strength” refers to the structural resistance to the radial compressive force(s) 845. FIG. 12D illustrates a diagrammatic cross-sectional view of the tube member 820, with arrows 855 indicating a tensile or expansive force. Thus, the term “tensile strength” refers to the structural resistance to the tensile force(s) 855.

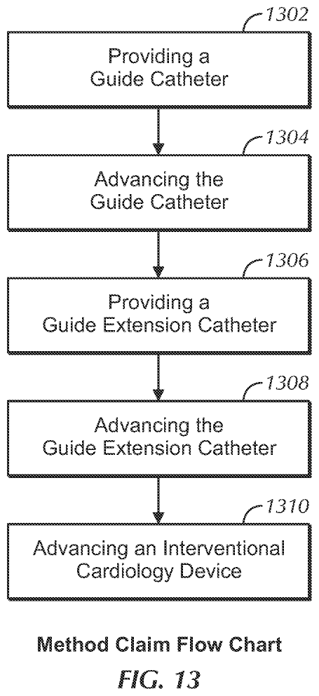

FIG. 13 illustrates a method for accessing a coronary artery, shielding the artery from abrasion or injury, and providing treatment to the artery. The method 1300 can include STEP 1302: PROVIDING A GUIDE CATHETER, which may include providing a guide catheter formed of polyurethane, for example, and can be shaped along its distal portion to facilitate advancement to a coronary ostium (or other region of interest within a patient's body). Any sized guide catheter may be provided, e.g., 6 F, 7 F, or 8 F guide catheter, where F is an abbreviation for the French catheter scale (a unit to measure catheter diameter (1 F=⅓ mm)).

The method 1300 includes STEP 1304: ADVANCING THE GUIDE CATHETER, which includes advancing the guide catheter through a blood vessel to a position adjacent to an ostium of the coronary artery. In some embodiments, the guide catheter can be inserted at a femoral artery or a radial artery and advanced through an aorta to a position adjacent to the ostium of a coronary artery. In some embodiments, the guide catheter may be guided by a guidewire, the guidewire optionally including a steerable and/or deflectable guidewire tip. The guide catheter may include radiopaque markers to communicate the location of the guide catheter as it advanced through the patient.

The method 1300 includes STEP 1306: PROVIDING A GUIDE EXTENSION CATHETER, wherein the guide extension catheter includes a push member and a tubular membrane wrapped about the push member. In some embodiments, the guide extension catheter includes a first reinforcement portion configured to maintain patency of a lumen. The tubular membrane may have no effective column strength, no effective radial strength, and no effective bend stiffness. In other words, any radial force, column force, or bend force will cause the tubular membrane to deflect, collapse, and/or bend, and the tubular membrane will provide no effective resistance of a radial, column, or bend force (see e.g., FIGS. 7A-7D). The tubular membrane may exhibit a tensile strength sufficient to prevent tearing of the walls of the tubular membrane and sufficient to prevent micro and/or macro injury to the vessel walls during advancement of an interventional cardiology device.

The method 1300 includes STEP 1308: ADVANCING THE GUIDE EXTENSION CATHETER, wherein the guide extension catheter is advanced through the guide catheter to a position where at least a portion of the tubular membrane extends distally beyond a distal end of the guide catheter and into the coronary artery. In some embodiments, the guide extension catheter is advanced by providing an advancement force on the push member.

The method 1300 includes STEP 1310: ADVANCING AN INTERVENTIONAL CARDIOLOGY DEVICE, including advancing the interventional cardiology device through the guide catheter and into a lumen defined by the tubular membrane and urging the tubular membrane to expand from a collapsed wrapped configuration to an expanded configuration. In some embodiments, advancing the interventional cardiology device into and through the lumen defined by the tubular membrane includes protecting an endothelium layer of the coronary artery from injury between the distal end of the guide catheter and a target tissue treatment area.

The present devices, systems, and methods provide or use a delivery tool to reduce arterial injury caused by (i) abrasion of the coronary endothelium during interventional device delivery, or (ii) coronary trauma/dissection caused by large delivery forces, active guide catheter back-up, or relatively rigid guide extension catheters. In contrast to existing more rigid guide extension catheters, the current devices are configured to optimize (1) coronary vessel protection and (2) lubricity of the entire intra-coronary delivery pathway.

The above Detailed Description includes references to the accompanying drawings, which form a part of the Detailed Description. The Detailed Description should be read with reference to the drawings. The drawings show, by way of illustration, specific embodiments in which the present devices, systems, and methods can be practiced. These embodiments are also referred to herein as “examples.”

The Detailed Description is intended to be illustrative and not restrictive. For example, the above-described examples (or one or more features or components thereof) can be used in combination with each other. One or more features of guide extension catheters 300, 300′, 500, 600, and/or 700, for example, may be interchangeable. For instance, guide extension catheter 300 and/or 310′ may include a distal reinforcement member comprising coil member 534 and/or distal reinforcement member 634. Certain elements, while numbered differently in separate figures, may be the same or substantially in size, shape, material composition, and/or configuration. For instance, radially-collapsible tubular membrane 310 may be similar, identical, or readily interchangeable with radially-collapsible tubular membrane 510 and/or 710. Likewise, push member 322 may be similar, identical, or readily interchangeable with push member 522 and/or 622. Other embodiments can be used, such as by one of ordinary skill in the art upon reviewing the Detailed Description and accompanying drawings. Also, various features or components have been or can be grouped together to streamline the disclosure. This should not be interpreted as intending that an unclaimed disclosed feature is essential to any claim. Rather, inventive subject matter can lie in less than all features of a disclosed embodiment. Thus, the following claims are hereby incorporated into the Detailed Description, with each example standing on its own as a separate embodiment:
In Example 1, a guide extension catheter positionable within a guide catheter and configured to receive an interventional device for insertion into vasculature can comprise a push member, a first reinforcement member in contact with the push member, and a radially-collapsible, tubular membrane in contact with the push member and the first reinforcement member. The tubular membrane is positioned distal to the first reinforcement member and is collapsed or wrapped about the push member prior to receiving the interventional device.
In Example 2, the guide extension catheter of Example 1 is optionally configured such that the tubular membrane has no effective column strength, no effective radial strength, no independent bend stiffness, and a tensile strength sufficient to prevent tearing during insertion of the interventional device.
In Example 3, the guide extension catheter of any one of Examples 1 or 2 is optionally configured such that a distal end of the push member extends distally of a distal end of the tubular membrane.
In Example 4, the guide extension catheter of Example 3 is optionally configured such that the distal end of the push member includes an atraumatic, guidewire-like distal end.
In Example 5, the guide extension catheter of Example 4 is optionally configured such that the atraumatic, guidewire-like distal end includes a tapered core surrounded by a coil.
In Example 6, the guide extension catheter of Example 5 is optionally configured such that the atraumatic, guidewire-like distal end receives and maintains a user-induced curve.
In Example 7, the guide extension catheter of any one of Examples 1 or 2 is optionally configured such that a distal end of the push member terminates at or adjacent to a distal end of the tubular membrane.
In Example 8, the guide extension catheter of any one or any combination of Examples 1-7 is optionally configured such that the push member tapers in one or more dimensions along a portion of its length.
In Example 9, the guide extension catheter of any one or any combination of Examples 1-8 is optionally configured such that the first reinforcement member includes a deployable loop.
In Example 10, the guide extension catheter of any one or any combination of Examples 1-8 is optionally configured such that the first reinforcement member defines a concave track leading into the tubular membrane and has a greater column strength and a greater radial strength than that of the tubular membrane.
In Example 11, the guide extension catheter of any one or any combination of Examples 1-10 is optionally configured such that the push member is in contact with an inner surface of the tubular membrane.
In Example 12, the guide extension catheter of any one or any combination of Examples 1-10 is optionally configured such that the push member is in contact with an outer surface of the tubular membrane.
In Example 13, the guide extension catheter of any one or any combination of Examples 1-10 is optionally configured such that the push member is affixed along a plane of an outer wall of the tubular membrane.
In Example 14, the guide extension catheter of any one or any combination of Examples 1-13 is optionally configured such that the tubular membrane is folded about the push member prior to receiving the interventional device.
In Example 15, the guide extension catheter of any one or any combination of Examples 1-14 is optionally configured such that the first reinforcement member is affixed to the push member and maintains patency of a lumen leading into a proximal end of the tubular membrane.
In Example 16, the guide extension catheter of any one or any combination of Examples 1-15 optionally further comprises a second reinforcement member disposed at a distal end of the tubular membrane. The second reinforcement member is configured to maintain patency of the lumen at the distal end of the tubular membrane.
In Example 17, the guide extension catheter of Example 16 is optionally configured such that the second reinforcement member selectively opens or closes the distal end of the tubular membrane.
In Example 18, the guide extension catheter of any one or any combination of Examples 1-17 is optionally configured such that the tubular membrane includes a wall thickness and an outer diameter, wherein a ratio of the outer diameter to the wall thickness ranges from 10:1 to 50:1, inclusive.
In Example 19, the guide extension catheter of any one or any combination of Examples 1-18 is optionally configured such that the tubular membrane is lubricious on one or both of an inner surface or an outer surface.
In Example 20, the guide extension catheter of any one or any combination of Examples 1-19 is optionally configured such that the tubular membrane is composed of a lubricious layer, a non-crosslinked polymer layer, and a crosslinked polymer layer.
In Example 21, a guide extension catheter for use with a guide catheter can comprise a radially-collapsible, tubular membrane defining a lumen when biased to an open position, the lumen including a central axis, and a push member in contact with the tubular membrane along the tubular member's entire length and extending proximal of the tubular membrane for slidably positioning the tubular membrane within and partially beyond a distal end of the guide catheter. The tubular membrane has no effective radial strength and is configured to collapse toward the central axis when subject to a radially-inward biasing force. The tubular membrane has a tensile strength sufficient to prevent tearing during insertion of an interventional cardiology device.
In Example 22, a method for accessing a coronary artery can comprise providing a guide catheter, advancing the guide catheter through a blood vessel to a position adjacent to an ostium of the coronary artery, providing a guide extension catheter including a push member and a radially-collapsible, tubular membrane wrapped about the push member prior to receiving an interventional device, advancing the guide extension catheter through the guide catheter to a position where at least a portion of the tubular membrane extends distally beyond a distal end of the guide catheter and into the coronary artery, and advancing an interventional cardiology device through the guide catheter and into a lumen defined by the tubular membrane, including urging the tubular membrane to expand from a collapsed configured to an expanded configuration.
In Example 23, the method of Example 22 is optionally configured such that advancing the interventional cardiology device into and through the lumen defined by the tubular membrane includes protecting an endothelium layer of the coronary artery from injury between the distal end of the guide catheter and a target tissue treatment area.

Certain terms are used throughout this patent document to refer to features or components. Different people may refer to the same feature or component by different names. This patent document does not intend to distinguish between components or features that differ in name but not in function.

The scope of the present devices, systems, and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, system, or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A guide extension catheter positionable within a guide catheter and configured to receive an interventional device for insertion into vasculature, the guide extension catheter comprising:
a push member;
a first reinforcement member in contact with the push member; and
a radially-collapsible, tubular membrane in contact with the push member and the first reinforcement member, the tubular membrane positioned distal to the first reinforcement member and collapsed or wrapped about the push member prior to receiving the interventional device.

2. The guide extension catheter according to claim 1, wherein the tubular membrane has no effective column strength, no effective radial strength, no independent bend stiffness, and a tensile strength sufficient to prevent tearing during insertion of the interventional device.

3. The guide extension catheter according to claim 1, wherein a distal end of the push member extends distally of a distal end of the tubular membrane.

4. The guide extension catheter according to claim 3, wherein the distal end of the push member includes an atraumatic, guidewire-like distal end.

5. The guide extension catheter according to claim 4, wherein the atraumatic, guidewire-like distal end includes a tapered core surrounded by a coil.

6. The guide extension catheter according to claim 5, wherein the atraumatic, guidewire-like distal end is configured to receive and maintain a user-induced curve.

7. The guide extension catheter according to claim 1, wherein a distal end of the push member terminates at or adjacent to a distal end of the tubular membrane.

8. The guide extension catheter according to claim 1, wherein the push member tapers in one or more dimensions along a portion of its length.

9. The guide extension catheter according to claim 1, wherein the first reinforcement member includes a deployable loop.

10. The guide extension catheter according to claim 1, wherein the first reinforcement member defines a concave track leading into the tubular membrane and has a greater column strength and a greater radial strength than that of the tubular membrane.

11. The guide extension catheter according to claim 1, wherein the push member is in contact with an inner surface of the tubular membrane.

12. The guide extension catheter according to claim 1, wherein the push member is in contact with an outer surface of the tubular membrane.

13. The guide extension catheter according to claim 1, wherein the push member is affixed along a plane of an outer wall of the tubular membrane.

14. The guide extension catheter according to claim 1, wherein the tubular membrane is configured to be folded about the push member prior to receiving the interventional device.

15. The guide extension catheter according to claim 1, wherein the first reinforcement member is affixed to the push member and configured to maintain patency of a lumen leading into a proximal end of the tubular membrane.

16. The guide extension catheter according to claim 15, further comprising a second reinforcement member disposed at a distal end of the tubular membrane, the second reinforcement member configured to maintain patency of the lumen at the distal end of the tubular membrane.

17. The guide extension catheter according to claim 16, wherein the second reinforcement member is configured to selectively open or close the distal end of the tubular membrane.

18. The guide extension catheter according to claim 1, wherein the tubular membrane includes a wall thickness and an outer diameter, wherein a ratio of the outer diameter to the wall thickness ranges from 10:1 to 50:1, inclusive.

19. The guide extension catheter according to claim 1, wherein the tubular membrane is lubricious on one or both of an inner surface or an outer surface.

20. The guide extension catheter according to claim 1, wherein the tubular membrane is composed of a lubricious layer, a non-crosslinked polymer layer, and a crosslinked polymer layer.

21. A guide extension catheter for use with a guide catheter, comprising:
a radially-collapsible, tubular membrane defining a lumen when biased to an open position, the lumen including a central axis; and
a push member in contact with the tubular membrane along the tubular member's entire length and extending proximal of the tubular membrane for slidably positioning the tubular membrane within and partially beyond a distal end of the guide catheter,
wherein the tubular membrane has no effective radial strength and is configured to collapse toward the central axis when subject to a radially-inward biasing force, the tubular membrane including a tensile strength sufficient to prevent tearing during insertion of an interventional cardiology device.

22. A method for accessing a coronary artery, the method comprising:
providing a guide catheter;
advancing the guide catheter through a blood vessel to a position adjacent to an ostium of the coronary artery;
providing a guide extension catheter, the guide extension catheter including:
a push member, and
a radially-collapsible, tubular membrane wrapped about the push member prior to receiving an interventional device;
advancing the guide extension catheter through the guide catheter to a position where at least a portion of the tubular membrane extends distally beyond a distal end of the guide catheter and into the coronary artery; and
advancing an interventional cardiology device through the guide catheter and into a lumen defined by the tubular membrane, including urging the tubular membrane to expand from a collapsed configured to an expanded configuration.

23. The method of claim 22, wherein advancing the interventional cardiology device into and through the lumen defined by the tubular membrane includes protecting an endothelium layer of the coronary artery from injury between the distal end of the guide catheter and a target tissue treatment area.
